(19) United States
(12) Patent Application Publication
(10) Pub. No.: US 2016/0310188 A1
(43) Pub. Date: Oct. 27, 2016

(54) Devices and Methods for Sacroiliac Joint Arthrodesis and Fixation

(71) Applicant: Trinity Orthopedics, LLC, San Diego, CA (US)

(72) Inventors: James F. Marino, San Diego, CA (US); Jamil Elbanna, San Diego, CA (US)

(21) Appl. No.: 15/138,782

(22) Filed: Apr. 26, 2016

Related U.S. Application Data
(60) Provisional application No. 62/153,391, filed on Apr. 27, 2015.

(51) Int. Cl. A61B 17/86; A61F 2/30; A61F 2/28; A61F 2/46; A61B 17/16; A61B 17/88

(52) U.S. Cl. CPC A61B 17/864; A61B 17/1659; A61B 17/8816; A61B 17/8897; A61F 2/28; A61F 2/4601; A61F 2/30988; A61F 2002/2835; A61F 2002/30995

(57) ABSTRACT

The method includes placing a first guide pin within a sacroiliac joint. The first guide pin is blunt-tipped and flexible such that the first guide pin takes a non-linear course along at least a portion of the sacroiliac joint. The method includes inserting a reamer over the first guide pin. The reamer has a flexible and cannulated shaft configured to follow the non-linear course taken by the first guide pin. The method includes reaming articular soft tissue and periarticular bone from both the ilium and the sacrum using the reamer and collecting reamed material within the shaft, removing the reamer and the reamed material resulting in a non-linear void coursing generally along the at least a portion of the sacroiliac joint, and advancing material into the non-linear void.

### CROSS-REFERENCE TO PRIORITY DOCUMENT

This application claims priority under 35 U.S.C. §119(e) U.S. Provisional patent application Ser. No. 62/153,391, filed on Apr. 27, 2015, and entitled “Devices and Method for Sacroiliac Joint Arthrodesis,” which is incorporated by reference herein in its entirety.

### BACKGROUND

The sacroiliac joint is a large diarthroidal joint, having hyaline cartilage. The joint has relatively congruent convoluted surfaces that normally move minimally with respect to each other. Several clinical studies have estimated the incidence of sacroiliac joint discomfort in patients with low back pain to be between about 22% to about 25% and the incidence of low back pain in patients who have had lumbar fusion surgery to be as high as 40% to 45%.

### SUMMARY

In one aspect, disclosed is a method of sacroiliac joint arthrodesis. The method includes placing a first guide pin within a sacroiliac joint. The first guide pin is blunt-tipped and flexible such that the first guide pin takes a non-linear course along at least a portion of the sacroiliac joint. The method includes inserting a reamer over the first guide pin. The reamer has a flexible and cannulated shaft configured to follow the non-linear course taken by the first guide pin. The method includes reaming articular soft tissue and periarticular bone from both the ilium and the sacrum using the reamer and collecting reamed material within the shaft, removing the reamer and the reamed material resulting in a non-linear void coursing generally along the at least a portion of the sacroiliac joint, and advancing material into the non-linear void.

The first guide pin can be inserted between subchondral or periarticular plates of the sacroiliac joint from a relatively posterior-medial to a relatively anterior-lateral direction. The first guide pin can be deflected by higher density periarticular bone and remain generally within the sacroiliac joint composed of relatively lower density articular soft tissue. The first guide pin can be inserted in a percutaneous manner using fluoroscopic visualization through a cannulated guide. The method can further include inserting an obturator having a shaft extending through a thin-walled cannula to at least a posterior extent of the non-linear void. The shaft of the obturator and the thin-walled cannula can be flexible and configured to follow the non-linear course taken by the first guide pin. The method can further include removing the obturator leaving the thin-walled cannula in position. Advancing material into the non-linear void can include advancing the material through the thin-walled cannula. The material can be advanced from within the thin-walled cannula while the thin-walled cannula is withdrawn from the non-linear void. The material advanced into the non-linear void can be selected from the group consisting of bone graft, bone graft substitute, cancellous bone, osteo-proliferative material, osteo-inductive material, and osteo-conductive material.

The method can further include fixing the sacroiliac joint with a fixation element with or without compression. Fixing the sacroiliac joint can include advancing a generally linear guide pin in a posterior-lateral to anterior-medial direction and placing the linear guide pin across at least a portion of the sacroiliac joint. The linear guide pin can be advanced from an osseous entry point, just caudal and lateral to the posterior superior lateral iliac ala, through the ilium and the sacroiliac joint and into the sacral ala and body, cranial to the first sacral foramen and caudal to the lumbosacral disc space on the ipsilateral side of the sacrum. The method can further include exchanging the linear guide pin with a blunt guide pin toward, but not through the anterior sacral cortex along a vector that intersects the anterior sacral cortex between the mid-sagittal plane and a parasagittal plane generally defined by medial margins of the sacral foramina. The method can further include inserting a cannulated reamer over the blunt guide pin to create a generally linear tract through the ilium, sacroiliac joint and a portion of the sacrum. The method can further include advancing at least one cannulated dilator tool over the blunt guide pin into the body of the sacrum but not through the anterior wall of the sacral body, and inserting the fixation screw for fixation. The method can further include advancing a cannulated burr through the anterior wall of the sacral body, forming an anterior sacral cortical defect lateral to the mid-sagittal plane and medial to the sacral foramina. The cannulated burr can be relatively blunt in profile, can have fine teeth or a diamond dust coated surface with or without a depth stop to minimize risk of injuring soft tissue structures anterior the sacrum.

The fixation screw can include a proximal segment having a first thread form having a first pitch; a distal segment having a second thread form having a second pitch that is coarser than the first pitch; an intervening, non-threaded central segment; a cannulated bore extending through at least a portion of the distal segment of the fixation screw; and a blunt, non-threaded post projecting from the distal segment. The post can be configured to insert through the anterior sacral cortical defect.

The method can further include inserting the distal segment of the fixation screw just short of the anterior sacral cortex and leaving the proximal segment of the fixation screw immediately adjacent to the posterior/lateral iliac cortex. The first thread form of the proximal segment can engage the ilium and the second thread form of the distal segment can engage the sacrum. The intervening non-threaded central segment can span the articular margins. The method can further include advancing material into at least a portion of the cannulated bore, the material selected from the group consisting of cancellous bone, osteo-proliferative, osteo-inductive, and/or osteo-conductive material. Advancing material can include using a delivery element configured to couple with at least a portion of the proximal segment of the fixation screw to maintain net-zero displacement force on the fixation screw as the material is advanced into the cannulated bore. The fixation screw can be radiolucent for improved radiographic fusion healing assessment.

In some variations, one or more of the following can optionally be included in any feasible combination in the above methods, apparatus, devices, and systems. More details of the devices, systems and methods are set forth in the accompanying drawings and the description below. Other features and advantages will be apparent from the description and drawings.

### BRIEF DESCRIPTION OF THE DRAWINGS

These and other aspects will now be described in detail with reference to the following drawings. Generally speaking, the figures are not to scale in absolute terms or comparatively but are intended to be illustrative. Also, relative placement of features and elements may be modified for the purpose of illustrative clarity.

FIG. 1A and FIG. 1B show perspective views of an implementation of a sacroiliac screw configured to provide compression across a sacroiliac joint;

FIG. 1C shows a proximal end view of the screw of FIGS. 1A-1B;

FIG. 2 shows a side exploded view of the dual pitch sacroiliac screw of FIGS. 1A-1C;

FIG. 3 shows a cross-sectional side view of the non-threaded central segment;

FIG. 4 shows a perspective proximal end view of the distal threaded segment;

FIG. 5A and FIG. 5B show perspective proximal and distal end views, respectfully of the proximal threaded segment;

FIG. 6A shows a perspective view of an interrelated implementation of a sacroiliac screw configured to provide compression across a sacroiliac joint;

FIG. 6B and FIG. 6C show end views of an interrelated implementation of a sacroiliac screw configured to provide compression across a sacroiliac joint;

FIGS. 7A-1 through 7A-5 shows side views of various lengths uni-body sacroiliac screws of FIG. 6;

FIGS. 7B-1 through 7B-5 show side views of various lengths uni-body sacroiliac screws of FIG. 6;

FIGS. 7C-1 through 7C-5 show perspective end views of various lengths uni-body sacroiliac screws of FIG. 6;

FIGS. 8-1 through 8-5 show the uni-body sacroiliac screws of FIG. 6 having an anterior sacral cortical post;

FIG. 9 shows a cross-sectional view of an implementation of a uni-body implant of FIGS. 8-1 through 8-5 having an anterior sacral cortical post;

FIGS. 10-1 through 10-5 show various anterior sacral cortical posts having differing lengths configured to fit the uni-body sacroiliac screws of FIG. 6;

FIG. 11A shows a posterior entry point for a guide pin to be placed within the sacroiliac joint for reaming;

FIG. 11B shows a lateral view of the sacrum showing the convoluted sacral articular surface;

FIGS. 11C-11D show transverse cross-sections taken along a plane extending medial-lateral and anterior-posterior along a section of the hip region in which a guide pin is inserted to a posterior extent of the sacroiliac joint (FIG. 11C) and a non-linear void courses along at least a portion of the sacroiliac joint (FIG. 11D);

FIG. 12A shows an approximate entry point for a guide pin that can course from posterolateral to anteromedial across the sacroiliac joint for fixation;

FIG. 12B shows a general location for an anterior sacral cortical device created by a burr for inhibiting shear movement upon implantation of sacroiliac screw;

FIG. 12C shows a generally orthogonal orientation of a sacroiliac screw relative to sacroiliac joint reaming and bone grafting.

It is to be understood that implants described herein may include features not necessarily depicted in each figure.

### DETAILED DESCRIPTION

Numerous methods for treating patients with chronic sacroiliac joint pain have been proposed that involve open procedures for fixation or the disruption of the articular cartilage, bone grafting, and internal fixation. Recent advances in fixation include porous rods or cannulated, fenestrated screws that are placed percutaneously and extend across the sacroiliac joint in several locations. At least one of these methods employs a cartilage and subchondral plate disruption that extends radially from the axis of dissection crossing the adjacent joint endplates. Most articular surgical synostosis (i.e. bone fusions) are effected by attempting to rigidly fix and frequently compress the to be “fused” joint with some form of internal fixation after denuding articular cartilage and subchondral bone from adjacent formerly articulating surfaces and replacing some of these resected tissues with a bone bridging material, such as autologous cancellous bone.

Described herein are devices, systems and methods for sacroiliac arthrodesis, fusion and/or stabilization using a percutaneous or mini-open surgical procedure. In some implementations, the methods involve denuding the dense material of the sacroiliac joint, cartilage, fibrous tissue, and periarticular bone creating a non-linear bleeding channel in which bone graft or bone graft substitutes are subsequently inserted. Subsequent fixation of the denuded joint using fixation screws or compression screws can be implemented. Compression across the joint can be achieved by a screw having a disparity in pitch between thread form segments. The devices, systems and methods described herein provide for minimally-invasive preparation of the sacroiliac joint for arthrodesis via percutaneous partial joint resection with or without percutaneous fixation providing joint fixation and/or compression, percutaneous deposition of osteo-conductive material across the joint line, enhanced stability of the implant through pen-implant bone impaction and anterior sacral cortical fixation, and radiolucent implant(s) for improved radiographic fusion healing assessment.

Methods of Sacroiliac Joint Arthrodesis

Any of the steps of the procedures described herein can be performed under fluoroscopic visualization or image intensifier imaging. Further, any of the procedures described herein can be performed in a percutaneous or mini-open manner.

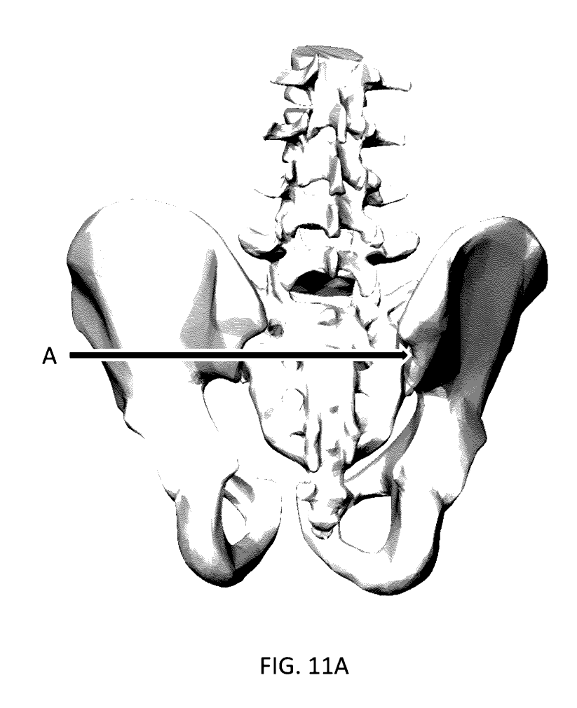

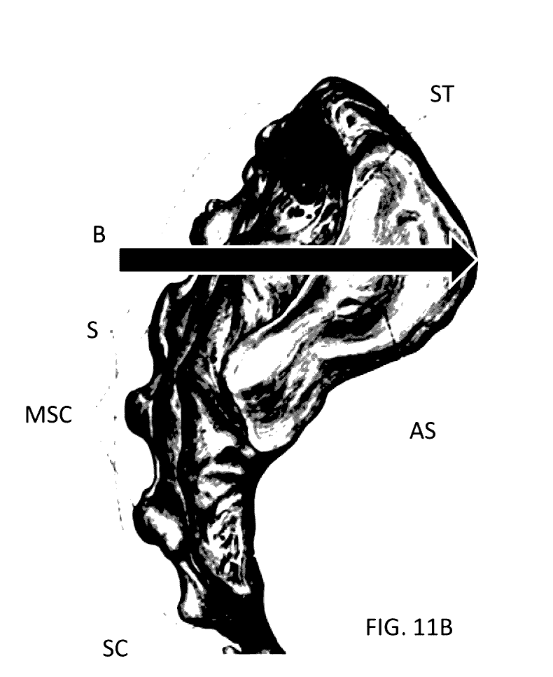

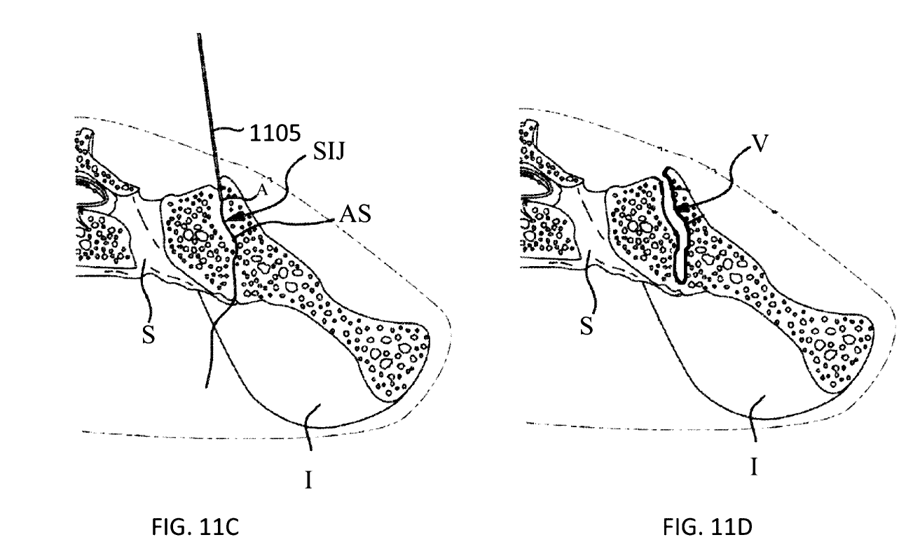

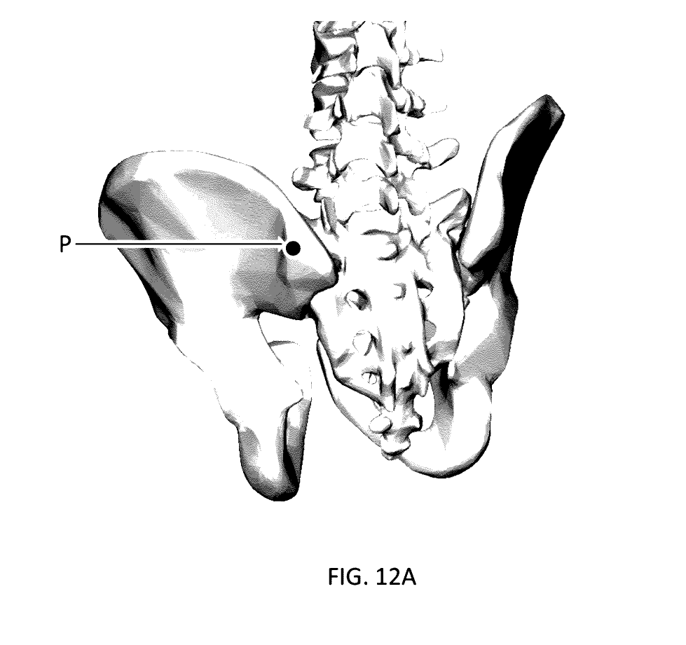

In a first implementation and as shown in FIGS. 11A-11B, the sacroiliac joint can be denuded using non-linear reaming along the joint line. A guide pin can be placed within the sacroiliac joint between the subchondral plates at posterior entry point A (see FIG. 11A and FIG. 11C). The guide pin can be positioned from a relatively posterior-medial to a relatively anterior-lateral direction. FIG. 11B shows a lateral view of the sacrum S showing the convoluted sacral articular surface AS, the sacral tuberosity ST, the median sacral crest MSC, and the sacral cornu SC. The general orientation of the guide pin from a postero-medial to anterio-lateral along a nonlinear path within the sacroiliac joint is shown as arrow B in FIG. 11B. FIGS. 11C-11B show transverse cross-sections taken along a plane extending medial-lateral and anterior-posterior along a section of the hip region in which a guide pin 1105 is inserted to a posterior extent of the sacroiliac joint SIJ (FIG. 11C) and a non-linear void V courses along at least a portion of the sacroiliac joint (FIG. 11D).

The guide pin can be blunt-tipped and flexible. The sacroiliac joint is a convoluted joint such that the flexibility of the guide pin allows it to be advanced through the joint and get deflected by higher density periarticular bone and remain generally within the joint line composed of relatively lower density articular soft tissue. The small diameter of the guide pin can contribute to its overall flexibility as can the material of the guide pin. For example, the guide pin can be formed of a relatively flexible, super elastic metal such as Nitinol. It should be appreciated that the guide pin can be placed percutaneously within the fibrocartilaginous articular space between the non-planar sacral and iliac periarticular osseous features through a guide. For example, a rigid needle obturator can be inserted into the back of the sacroiliac joint and the flexible guide pin inserted through the obturator. Alternatively, a stiff guide pin can be placed to the opening of the sacroiliac joint and a cannulated obturator positioned down to the joint over the stiff guide pin. The obturator and stiff guide pin can be removed and the flexible guide pin inserted through the cannula into the joint. The flexible guide pin can take a non-linear course corresponding with the sacroiliac joint until it is placed within the sacroiliac joint between the subchondral or periarticular plates.

Once the flexible guide pin is inserted into the joint, a reamer can be inserted over the guide pin to perform non-linear reaming of the sacroiliac joint to remove cartilage, fibrous tissue, and periarticular bone thus, creating a non-linear bleeding channel. The reamer can have a flexible cannulated shaft such that it can follow the non-linear course taken by the flexible guide pin. The reamer can be used to ream articular soft tissue and periarticular bone from both the ilium and the sacrum. The cannulated reamer can have forward projecting and raked cutting surfaces configured to resect and collect the reamed material (i.e. articular cartilage and adjacent cortical and cancellous bone). Removing the reamer and the reamed material collected within the flexible and hollow reamer shaft results in a non-linear void generally coursing along the sacroiliac joint. The non-linear void can be subsequently filled with material. The material can include, but is not limited to, bone graft, bone graft substitutes, cancellous bone, such as in dowel or cylindrical form, osteo-proliferative, osteo-inductive, and/or osteo-conductive material. The insertion of material can be inserted through a cannula inserted through at least a portion of the void. In some implementations, upon removing the reamer and reamed material an obturator associated with a thin-walled cannula is inserted to a posterior extent of the non-linear void or into the non-linear void in the sacroiliac joint. The obturator can extend through a thin-walled cannula such that a distal tip of the obturator projects beyond a distal end of the cannula. The shaft extending through the cannula and the cannula can both be flexible such that they can course through the non-linear void created. The distal tip of the obturator need not be flexible and can be relatively rigid. The flexibility of the obturator shaft and cannula can be provided by material selection as well as structural characteristics. The shaft and the cannula can both be machined such as by laser cutting to create slits in the wall as is known in the art providing flexibility.

The subsequent insertion of material along the path of cartilage and subchondral bone resection can be provided for via extrusion delivery and compaction. The extrusion delivery and compaction can be from the distal end (i.e. distant end located away from surgeon operator) of the non-linear, thin-walled cannula upon removal of the obturator. The cannula can deliver material from within the cannula as it is withdrawn from the joint resection tract (e.g. anterior to posterior). The joint can then undergo fixation with a screw(s) or other relatively rigid form of fixation as described in more detail below.

Methods of Sacroiliac Joint Fixation

The sacroiliac joint following arthrodesis and induction of joint ossification can be further stabilized by the insertion of one or more fixation element(s), such as fixation screws, advanced across the sacroiliac joint. It should be appreciated, however, that the reaming and filling of the non-linear void with bone graft materials can be performed with or without subsequent fixation. Further, the sacroiliac joint fixation can incorporate any of a number of fixation elements including fixation screws known in the art as well as the sacroiliac screws described herein, which may or may not involve compression. Thus, the fixation can, but need not provide compression across the sacroiliac joint.

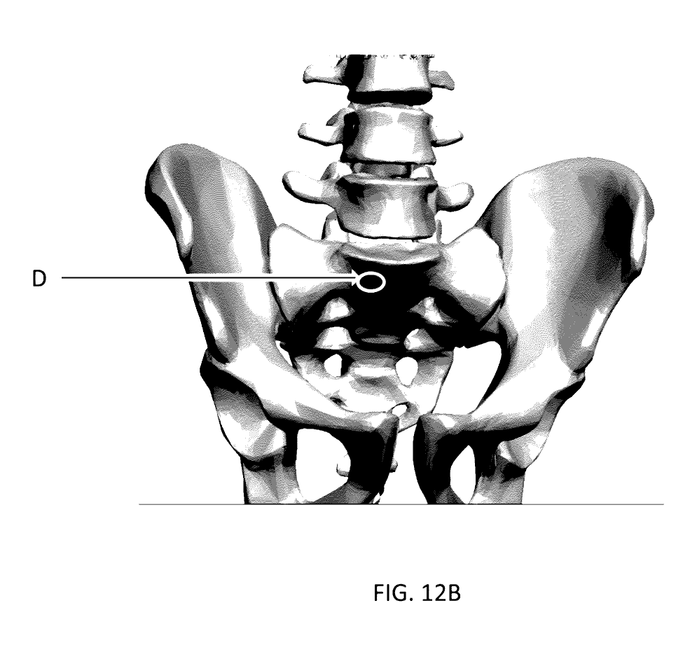

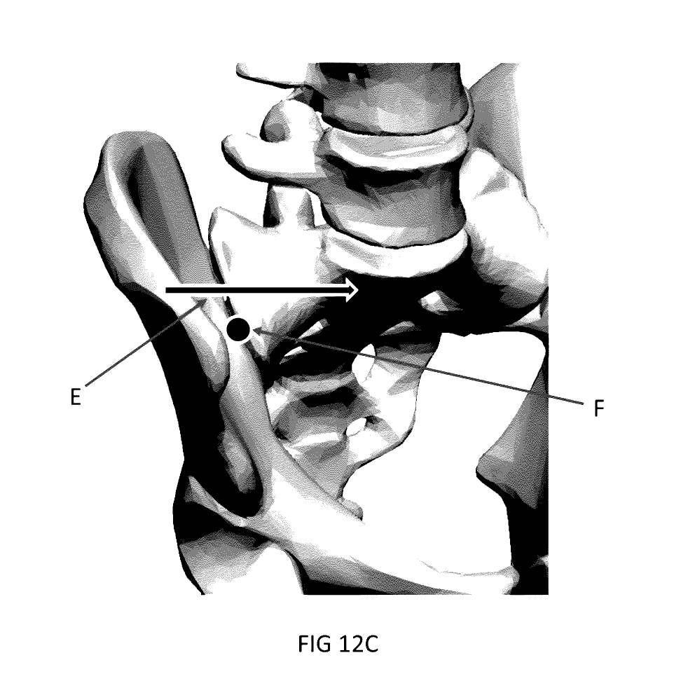

In a first implementation of a method of sacroiliac joint fixation, one or more guide pins can be advanced from the ilium through the sacroiliac joint and into the sacrum (e.g. from the posterior iliac ala into the sacral alar and body). One guide pin can be advanced from an osseous entry point, just caudal and lateral to the posterior superior lateral iliac ala, through the ilium and sacroiliac joint and into the sacral ala and body, cranial to the first sacral foramen and caudal to the lumbosacral disc space (on the ipsilateral side of the sacrum). FIG. 12A shows an approximate entry point P for the guide pin that can course from posterolateral to anteromedial across the sacroiliac joint. A second guide pin can be advanced along a generally parallel path to the first guide pin, entering the ilium posterior and inferior to the first guide pin and advancing linearly to an area of the sacrum that is caudal to the first sacral foramen and cranial to the second sacral foramen. Unlike the guide pin 1105 used for sacroiliac joint arthrodesis that is flexible and advanced from a generally posterior-medial to a generally anterior-lateral direction along a non-linear path of the sacroiliac joint SIJ, the guide pins used in the insertion of fixation screws are generally linear and advanced from a generally posterior-lateral to anterior-medial direction across the sacroiliac joint. The initial guide pin can be sharp and bone penetrating such that it can be advanced toward, insert into, but not through the anterior wall of the sacral body (i.e. the anterior sacral cortex). The initial guide pin(s) are advanced along a vector that intersects the anterior sacral cortex between the midline (i.e. mid-sagittal plane) and a parasagittal plane generally defined by medial margins of the sacral foramina.

Once these guide pins are positioned, either percutaneously or via a mini-open surgical procedure, a cannulated obturator or series of sequential obturators or progressively larger diameter cannulas can be advanced through the incised skin and fascia to dilate the soft tissue envelope along the guide pin(s) axis to the level of the iliac cortical bone entry. The largest thin-walled cannula can be advanced and temporarily secured with the soft tissue envelope, an operator's grasp, an external brace or clamp, and/or via small diameter bone fixation pins that can be delivered through channels associated with the side wall of the cannula.

Either before and after initial coaxial reaming over the sharp bone penetrating guide pin (exercising care not to advance the guide pin through the anterior sacral cortex), a blunt-tipped guide pin can be exchanged for the sharp bone penetrating guide pin. This blunt-tipped guide pin can be advanced within the sacrum short of penetrating the anterior sacral cortex and serve to guide subsequent cannulated instrumentation along the predefined surgical path with reduced risk for anterior sacral cortical penetration and thus, reduce the risk of injuring intra-pelvic structures.

With the blunt guide pin in place and the cannula relatively fixed or held in a position that is generally coaxial with the guide pin(s), the cannulated obturator(s) or smaller diameter cannula(s) can be removed. Subsequently, a cannulated drill or cannulated trocar tip can be advanced within the cannula and over the blunt-tipped guide pin to penetrate and remove and/or displace bone radially along the axis of the guide pin(s). A trephine to harvest cylindrical bone or the cannulated drill to harvest morselized bone can be advanced along the course or previous course of the blunt tipped guide pin should it be removed for the trephine. A trocar tip or drill can be used to advance along this same guide pin axis across the sacroiliac joint and into the subchondral aspect of the lateral sacral alar.

A radial or sequential radial impaction reamer(s) or dilator(s) can be used to radially displace bone (primarily cancellous) along all or a portion of the axis of the guide pin or former tract, to radially impact cancellous bone. This increases cancellous bone density for improved subsequent sacroiliac screw thread purchase. Impaction reaming and dilation can be performed using bullet-shaped tools having sequentially larger and larger diameter to push and compact bone radially away from the guide pin axis. Sequentially larger diameter impaction reaming over the blunt-tipped guide pin in the sacral alar can enhance sacral bone density and thus, enhance distal screw purchase within what would otherwise be a relatively cancellous portion of the sacral body. Previously removed or cadaveric allograft cancellous bone can be delivered into the tract for additional radial impaction.

The initial reaming and dilation can be limited to the ilium, the sacroiliac joint and the cancellous portion of the sacral body, but stop short of the anterior sacral wall due to the risk of damage to soft tissue structures anterior the sacrum. However, the cortex of the sacrum can provide the densest material for fixation element purchase. Thus, prior to insertion of a fixation element, such as the sacroiliac screws described herein, an anterior sacral cortical hole can be created using a relatively blunt, cannulated burr for insertion of at least a distal end region of the fixation element into the sacral cortex. The burr can be a fluted, non-aggressive or abrasive burr having fine teeth or a diamond dust coated surface with or without a depth stop to grind away at the bone while minimizing the risk of injuring soft tissue structures anterior the sacrum. The burr can be deployed along the guide pin axis (or axes) and into the anterior sacral cortical wall. The burr can be deployed with the intent of minimal depth of penetration sufficient to prepare a circular cortical defect and careful to avoid damaging soft tissue structures (e.g. vascular and neurologic) anterior to the sacrum. The anterior sacral cortical hole can be created lateral to the mid-sagittal plane and medial to the sacral foramina (reducing the risk of vascular and nerve injury).

An externally graduated tap that references off the lateral iliac cortex and/or the protective cannula can provide an indication of the optimized length of sacroiliac screw desired for implantation, which will be described in more detail below. For example, at least a portion of the sacroiliac screw can reside just short of the anterior sacral cortex while leaving a more proximal portion of the screw immediately adjacent to the posterior/lateral iliac cortex. At least a portion of the distal region of the sacroiliac screw can insert through the burred anterior sacral cortical hole to reduce implant migration under sacroiliac joint shear movement, which will also be described in more detail below. FIG. 12B shows a general location for anterior sacral cortical defect D created by a non-aggressive burr and eventually occupied by an intra-cortical post of a sacroiliac screw. FIG. 12C shows a generally orthogonal orientation of a sacroiliac screw relative to sacroiliac joint reaming and bone grafting. The general orientation of a sacroiliac screw is shown by arrow E and the general orientation of the non-linear path of the sacral joint reaming and grafting is shown at F.

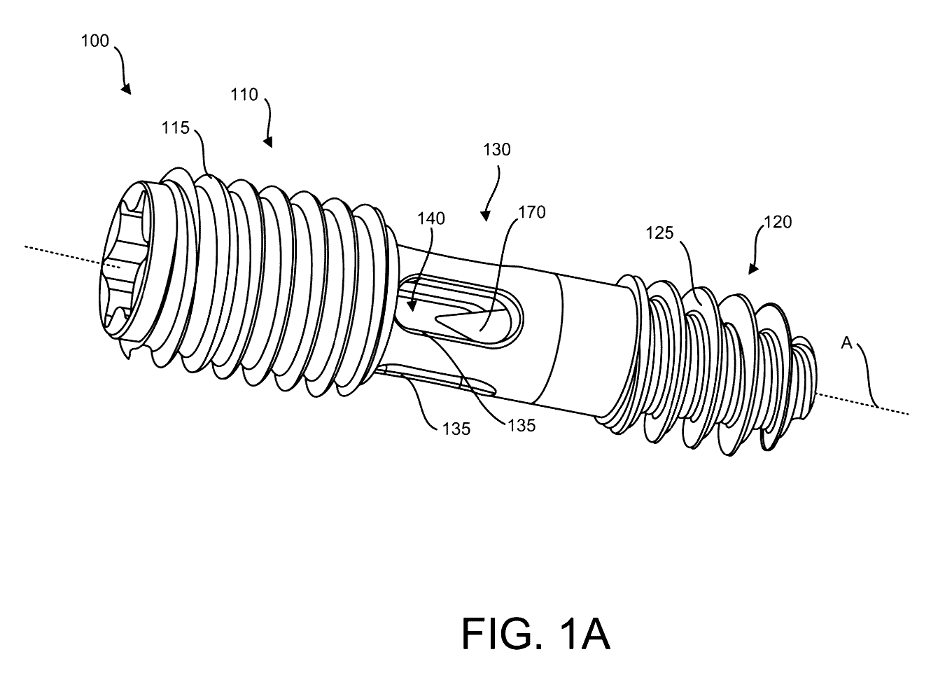

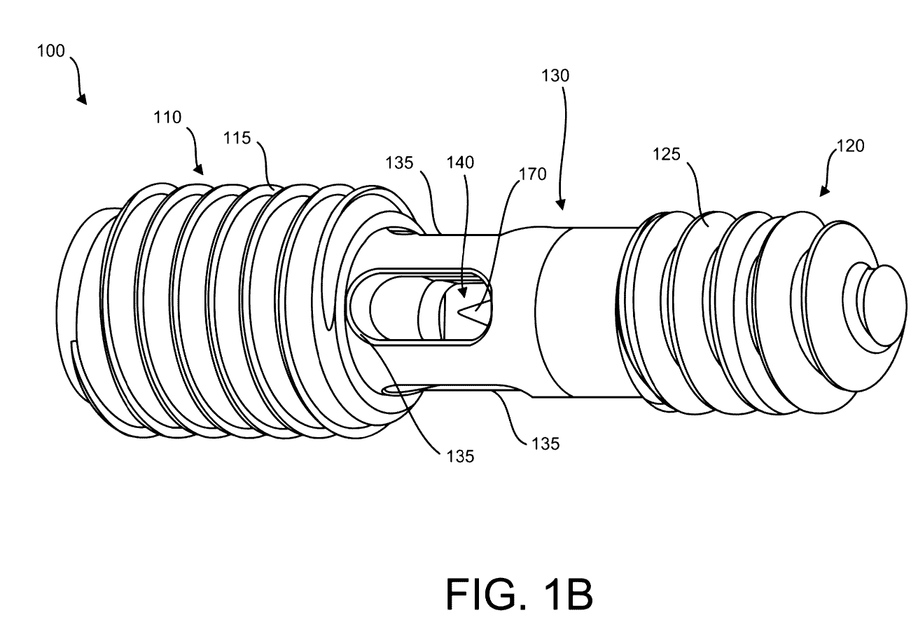

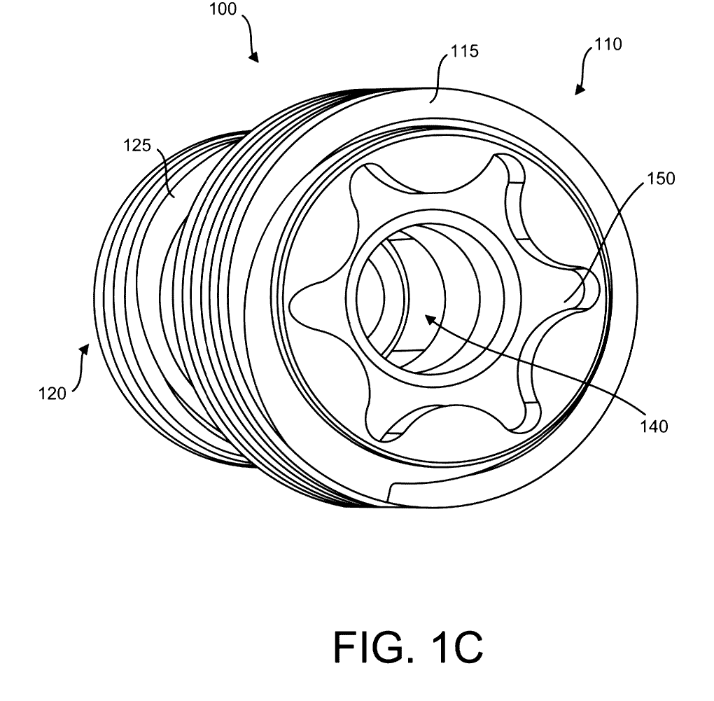

In some implementations, the fixation element delivered through the tract can provide compression across the sacroiliac joint. Compression across the joint can be achieved via a disparity in pitch between thread forms of the screw. FIGS. 1A-1B show perspective views of an implementation of a sacroiliac screw 100 configured to provide compression across a sacroiliac joint. The sacroiliac screw 100 can have a proximal segment 110 having a first thread form 115 and a distal segment 120 having a second thread form 125 and an intervening central segment 130. The central segment 130 can be non-threaded. The pitch of the first thread form 115 can be different from the pitch of the second thread form 125. For example, the pitch of the second thread form 125 can be coarser than the pitch of the first thread form 115. The disparity in pitch between the first and second thread forms 115, 125 can cause compression across the sacroiliac joint. The proximal thread form 115 can engage a first bone such as the ilium and the distal thread form 125 can engage a second bone such as the sacrum. The intervening non-threaded segment 130 can span the articular margins. Upon advancement into the bone and engagement of the thread forms 115, 125 with the two bones, tension can be placed on the screw 100. The proximal thread form 115 can have a more shallow pitch than the distal thread form 125. Further, the external diameter of the distal segment 120 can be smaller than the external diameter of the proximal segment 110.

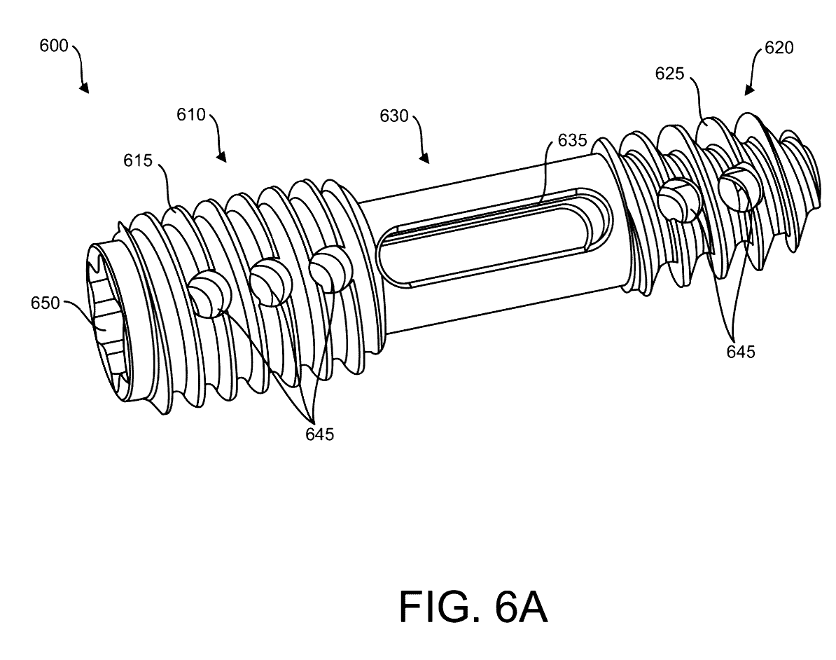

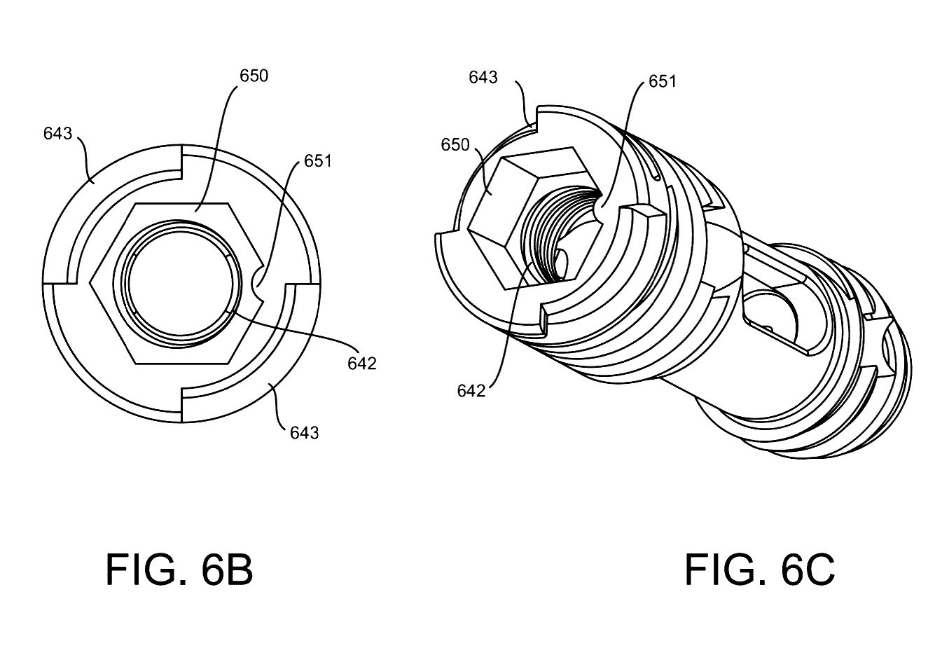

The trans-articular central segment 130 can have at least one side wall fenestration 135. The side wall fenestration 135 can provide for communication between the space outside the sacroiliac screw 100 with a cannulated bore 140 extending through at least a portion of the screw 100. In some implementations, the central segment 130 can have two or more side wall fenestrations 135. The two or more side wall fenestrations 135 can provide for osseous growth from one side of the screw 100 to the other. Additional fenestrations 145 can be formed in one or more side walls of the proximal or distal segments 110, 115 (see, for example, FIG. 6). These additional fenestrations 145 can also provide for communication between the cannulated bore 140 and the space outside the sacroiliac screw 100. The cannulated bore 140 and/or the fenestrations 135, 145 can be pre-filled, in part or in whole, with cancellous bone. Filling these spaces, which can be performed before, during and/or after implantation, can provide a relatively continuous path for one to bridge from ilium to sacrum as well as around and/or through the sacroiliac screw 100.

FIG. 1C shows a proximal end view of the screw 100 illustrating the cannulated bore 140 and a shallower pitch to the proximal thread form 115 compared to the steeper pitch thread form 115 of the distal segment 120. The proximal end of the screw 100 can also include a recessed screw drive 150. Further, the proximal segment 110 can include a feature that can be used, for example, to advance bone graft material into the cannulated bore 140. In some implementations, at least a portion of the cannulated bore 140 can include an internal thread form (see 642 of FIG. 6C, for example) or undercut configured to couple with another element to advance bone graft material into the cannulated bore 140 as will be described in more detail below. In some implementations, the proximal segment 110 can include an external feature or groove (see 643 of FIG. 6C, for example). It should be appreciated that the coupling features between the screw and the delivery element can vary. In some implementations, the coupling features include an external “push-rotate-lock” type of feature. In other implementations, the coupling feature incorporate corresponding thread forms.

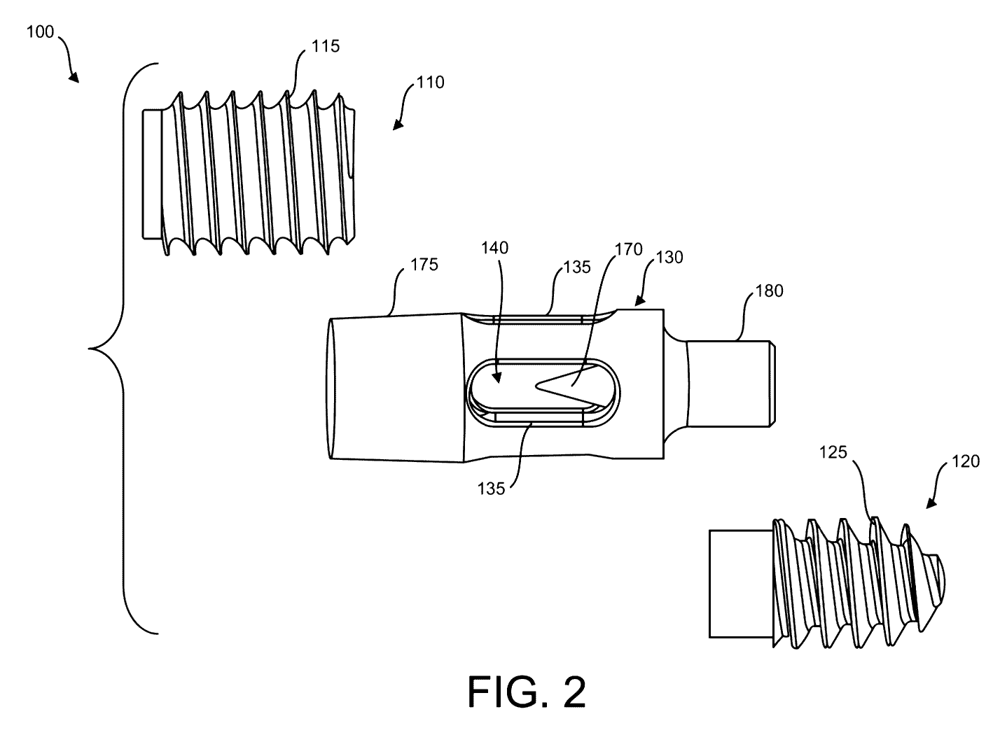

FIG. 2 shows a side exploded view of the dual pitch sacroiliac screw 100 having a proximal threaded segment 110, a distal threaded segment 120 and an intervening, central non-threaded segment 130. The screw 100 can include a dispersion element 170 extending within at least a region of the cannulated bore 140. The dispersion element 170 can be radially-directed or have an angled surface or surfaces relative to a long axis A of the screw 100. The dispersion element 170 can divert material within the bore 140 such as bone graft material, cancellous bone or other osteo-proliferative, and/or osteo-inductive, and/or osteo-conductive material towards or into at least one side wall fenestration 135. In some implementations, the dispersion element 170 has a generally conical form and is positioned adjacent to the fenestration(s) 135 of the central segment 130. The bone graft material can be delivered generally along the long axis A of the screw 100, such as from a proximal to a distal end within the cannulated bore 140. The dispersion element 170 can divert the material to and through the fenestrated side walls.

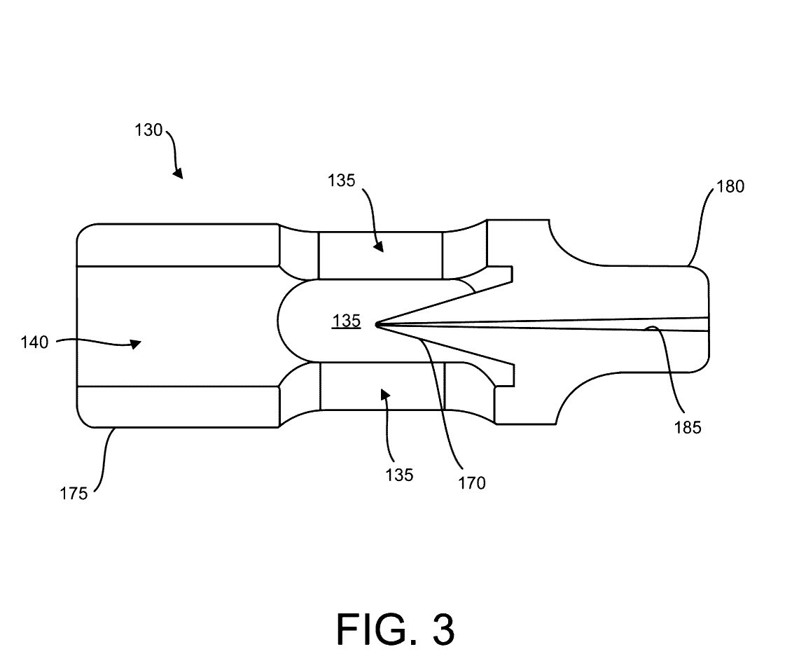

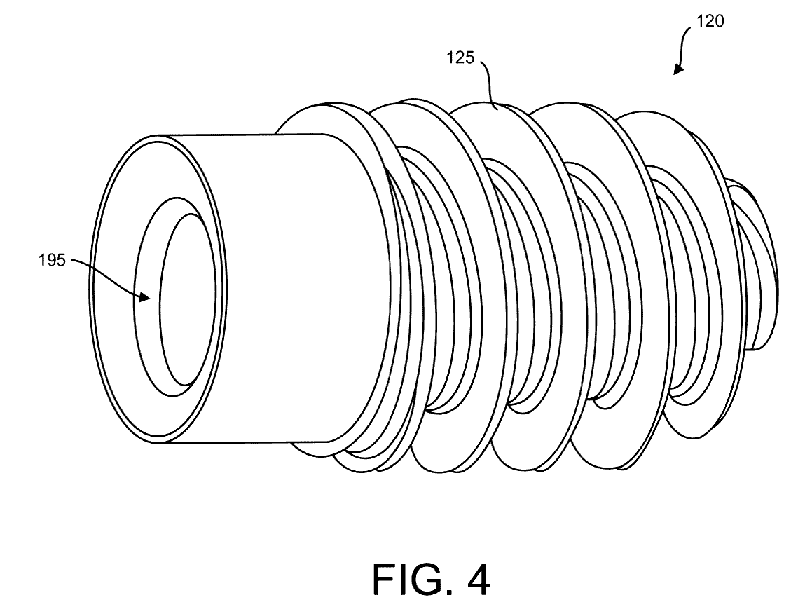

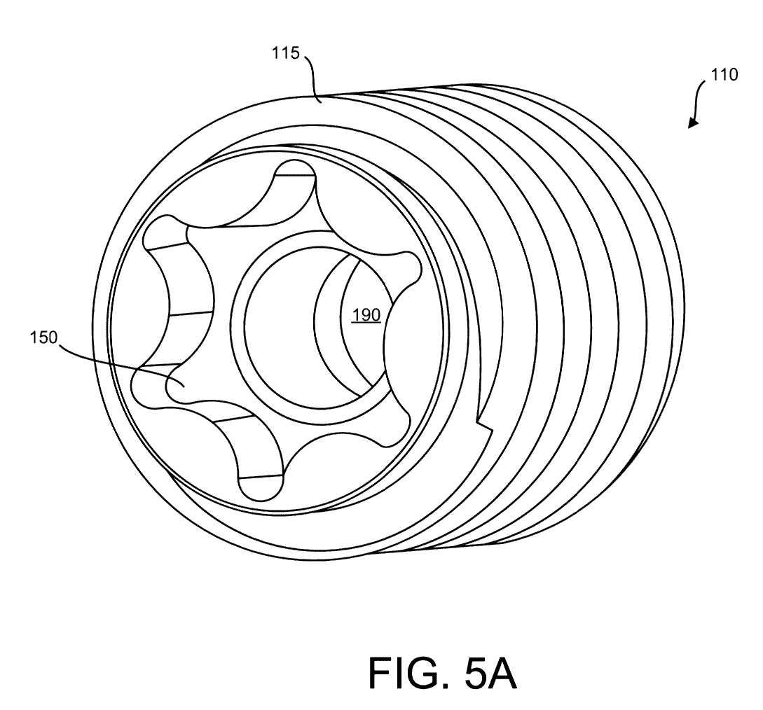

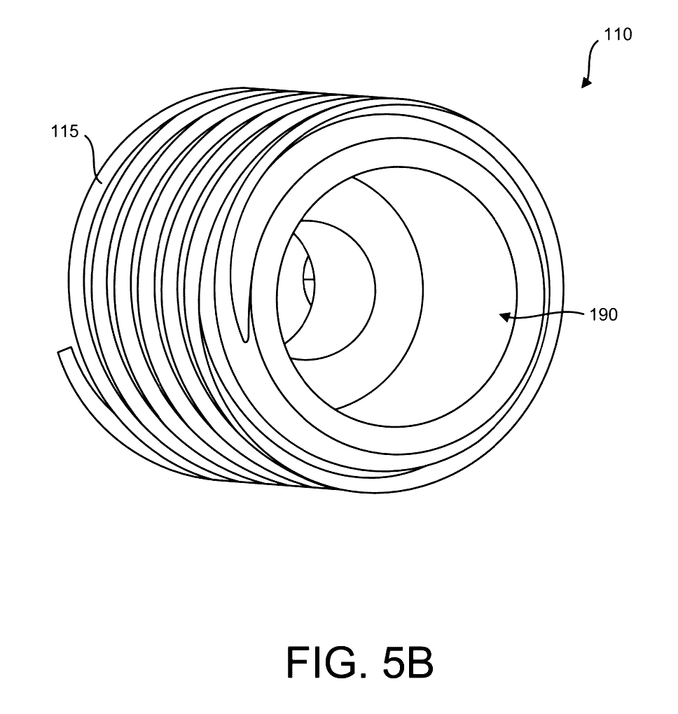

FIG. 3 shows a cross-sectional side view of the non-threaded central segment 130. FIG. 4 shows a perspective proximal end view of the distal threaded segment 120. FIGS. 5A-5B show perspective proximal and distal end views, respectfully of the proximal threaded segment 110. As best shown in FIG. 3, the cannulated bore 140 extends through at least a proximal end of the central segment 130 and in communication with sidewall fenestrations 135 of the central segment 130. The proximal end 175 of the central segment 130 can include an external trunnion or Morse taper. Similarly, the distal end 180 of the central segment 130 can include an external trunnion or Morse taper. The taper on the proximal end 175 is configured to wedge with at least a portion of an internal bore 190 in the proximal segment 110 such that internal bore 190 and cannulated bore 140 are confluent. Taper on the distal end 180 of the central segment 130 is configured to wedge with at least a portion of an internal bore 195 of the distal segment 120. The dispersion element 170 can include a cannulation 185 such that internal bore 195 and cannulated bore 140 can be confluent as well.

FIG. 6A shows a perspective view of an interrelated implementation of a sacroiliac screw 600 configured to provide compression across a sacroiliac joint. The screw 600 can be a uni-body or monolithic element having fenestrations 635 through a central segment 630 and one or more fenestrations 645 through one or both of the proximal segment 610 and distal segment 620. The fenestrations 635, 645 provide for communication between the space outside the sacroiliac screw 600 with a cannulated bore 640 extending through at least a portion of the screw 600. The proximal segment 610 can have a first thread form 615 and the distal segment 620 can have a second thread form 625 and the central segment 630 can be non-threaded. As with previous implementations, the pitch of the first thread form 615 can be different from the pitch of the second thread form 625, the disparity in pitch causing compression across the sacroiliac joint upon advancement of the screw 600. The proximal segment 610 thread form 615 can have a more shallow pitch than the distal thread form 625 such that tension is placed on the screw 600 when advancement of the threaded sections 615, 625 engaging the sacrum and ilium, respectively. As described above, these threaded segments 615, 625 of the screw 600 can be separated by the intervening non-threaded segment 630 that spans the former articular margins.

The screw 600 can have an “I” beam profile and the fenestrations 635, 645 a single plane of orientation. The “I” beam effect can be achieved by orienting material in optimized position to resist bending and shear loads. The extracorporeal orientation of an insertion driver to orient the implant's graft communications windows can optimize the shear strength of the screw under loading. The sacroiliac screw driver can be indexed, such as by an asymmetric geometry associated with the engagement features 650, mating the screw driver with the screw 600 such that an indicator(s) on the screw driver shaft or handle (e.g. the axis of the “T-handle” driver) provides an extracorporeal and visible reference that corresponds with a plane that is coplanar or orthogonal with the plane defined by the fenestration(s) 635 in the middle segment 630 of the sacroiliac screw 600. This indexing can optimize the orientation of the screws fenestration(s) 635 for mechanical strength and/or X-ray visualization.

FIGS. 6B-6C show proximal end views of the sacroiliac screw 600 incorporating a drive recess 650 on the proximal end of the proximal segment 610. The drive recess 650 can be a hex head recess or similar screw drive feature configured to engage with an insertional driver. The drive recess 650 can be indexed or incorporate an indexing feature 651. The indexing feature 651 can be positioned around the longitudinal axis of the screw 600 such that it allows for orientation extracorporeally of a screw driver shaft marking or handle relative to the position of one or more of the fenestrations 635, 645 of the screw 600.

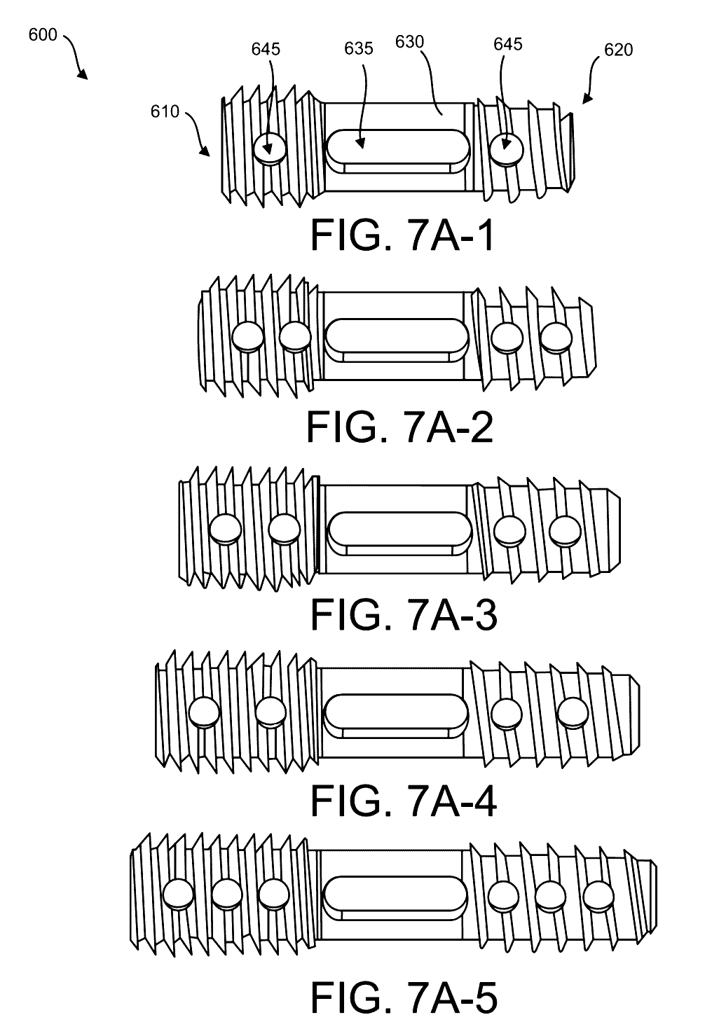

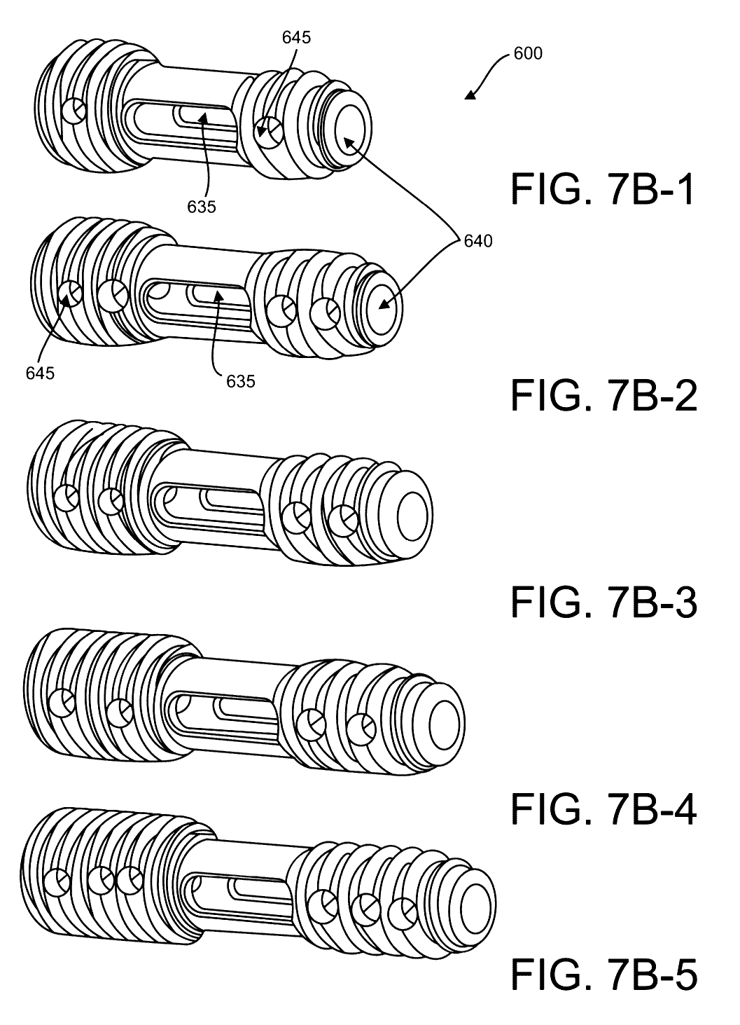

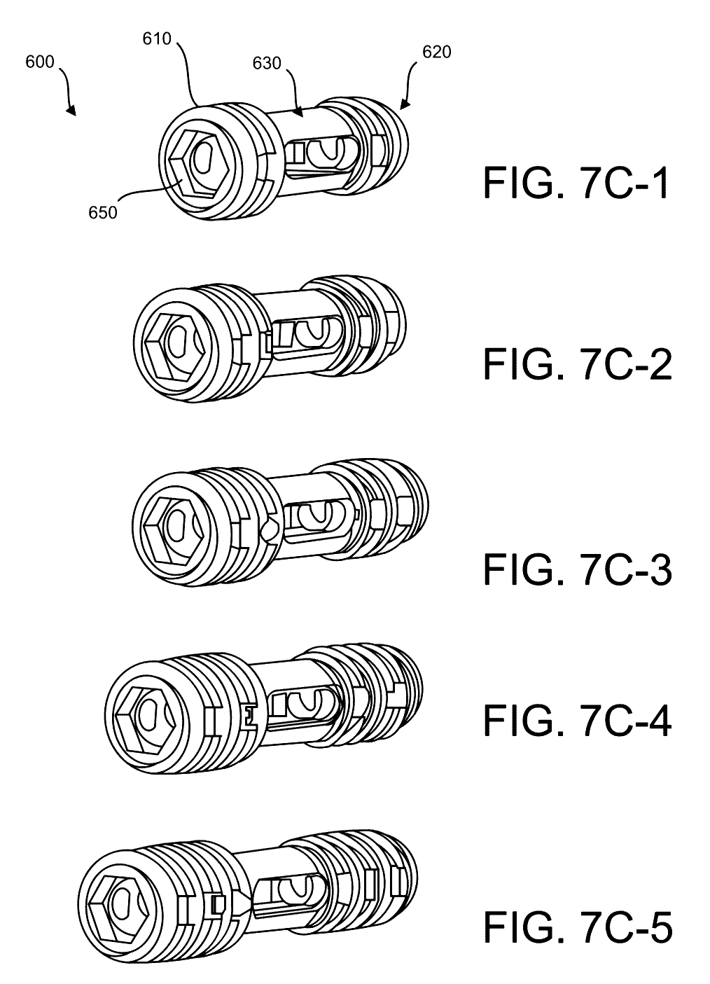

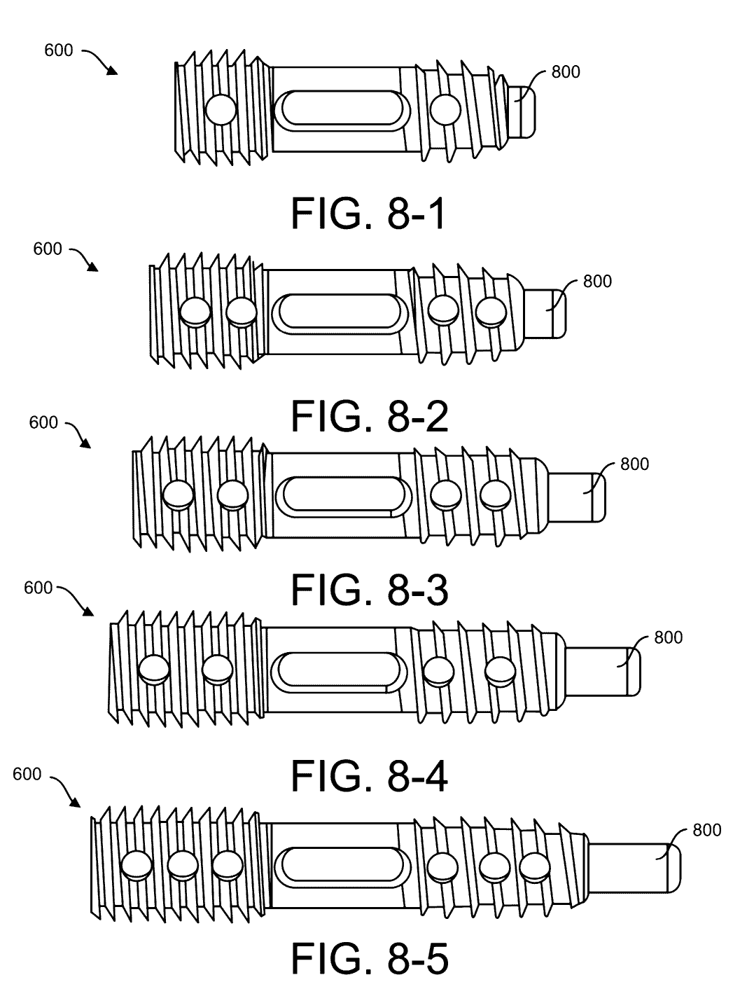

FIGS. 7A-1 through 7A-5 show side views and FIGS. 7B-1 through 7B-5 and FIGS. 7C-1 through 7C-5 show perspective end views of various lengths uni-body screws 600 that are cannulated and fenestrated. The constructs are shown without anterior sacral cortical wall posts. FIGS. 8-1 through 8-5 show the various constructs 600 having an anterior sacral cortical post 800. The post 800 can be modular such that it engages the screw by a snap fit, press fit, or threaded engagement between corresponding features in the cannulated bore 640 of the screw 600. The distally-ending post 800 need not be modular and can be a unitary portion of the screw 600. As mentioned above, the anterior sacral cortical post 800 can be inserted through the burred anterior sacral cortical hole to reduce implant migration under sacroiliac joint shear movement.

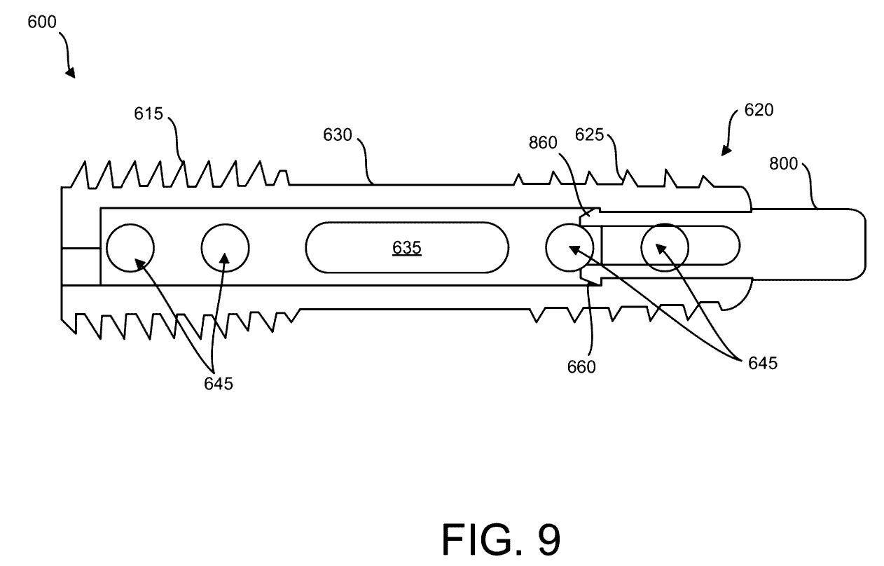

FIG. 9 shows an implementation of a uni-body implant 600 having an anterior sacral cortical post 800 extending through at least a region of the distal segment 620 of the screw 600. The post 800 can have a first region configured to insert or extending through at least a region of the distal segment 620 of the screw 600 and a second region configured to extend outside the cannulated bore 640. The first region of the post 800 can be slotted such that the slot 810 aligns with the fenestrations 645 of the screw 600 thereby preventing any blockage of the fenestrations 645 by the post 800 when the post 800 is inserted through the distal segment 620. The slot 810 can extend through the first region of the post 800 forming opposed legs. The legs can have a length sufficient to extend through at least a portion of the cannulated bore of the screw 600 such that an external feature 860 of the legs can engage with a corresponding internal feature 660 on a surface of the cannulated bore 640 of the screw. The external feature 860 of the legs can be a snap lock feature. When the external feature 860 is engaged with the internal feature 660 of the bore 640 the second region of the post 800 can extend outside the cannulated bore 640. This second region of the post 800 can be generally cylindrical, smooth, and having no slot. Thus, the slot 810 can terminate near the terminus of the cannulated bore 640 of the distal segment 620. The cylindrical second region of the post 800 can have a slightly larger outer diameter or dimension compared to the outer dimension of the opposed legs of the slotted first region such that a ledge or flange is formed between the second region and the first region. This ledge can abut and mate with the distal terminus of the screw 600 upon latching between the internal feature 660 and the external feature 860 (see FIG. 9). Generally, the region of the post 800 extending beyond the distal end of the screw 600 is a smooth, unthreaded, element configured to insert through a burred anterior sacral cortical hole to reduce implant migration under sacroiliac joint shear movement.

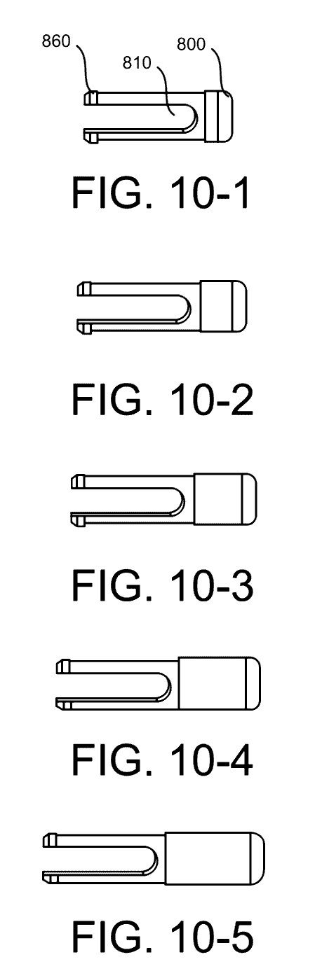

Because the screw 600 can vary in length as shown in FIGS. 7A-7C and FIG. 8-1 through FIG. 8-5, so too may the post 800. The post 800 can have differing lengths configured to fit the length of sacroiliac screw 600 and to extend beyond the screw 600 by increasing lengths. The increased length can be provided by an increasing the length of the second region as best shown in FIGS. 8-1 through 8-5 and FIGS. 10-1 through 10-5. As described above, this cylindrical second region of the post 800 can be configured to extend outside the cannulated bore 640 and thus, increased length in this region of the post 800 can result in an increased extension of the post 800 outside the bore 640. In some implementations, the length of the slot 810 and thus, the length of the opposed legs in the first region can be substantially the same and only the cylindrical second region of the post 800 has different lengths. The cylindrical second region of the post 800 can extend about 0.1 mm, 0.15 mm, 0.2 mm, 0.25 mm, 0.5 mm, 1.0 mm, 1.5 mm, 2.0 mm, 2.5 mm, 3.0 mm, 4.0 mm, 5.0 mm or other length beyond the distal end of the screw 600.

As mentioned above, upon creating the channel for fixation screw insertion including a burred anterior sacral cortical hole to reduce implant migration under shear movements, a tap and depth gauge can be used to provide an indication of the optimized length of sacroiliac screw desired for implantation. The depth gauge can be inserted through a central cannulation in the tap to determine the distance from the proposed final resting position of the sacroiliac screw 600 to the anterior cortex of the sacrum. An appropriate length anterior sacral cortical post 800 can be secured to the distal aspect of the sized sacroiliac screw 600. It should be appreciated, however, that the screw 600 need not be modular and have a distally-extending feature coupled to its distal segment that is configured to insert into the anterior sacral cortical defect or hole. The screw 600 can then be inserted through the channel such that the distal segment 620 of the screw 600 resides just short of the anterior sacral cortex while leaving the proximal segment 610 of the screw 600 immediately adjacent to the posterior/lateral iliac cortex. At least a portion of the cortical post 800 extending beyond a distal segment 620 of the screw 600 can insert through the burred anterior sacral cortical hole. As mentioned above, the thread forms of the distal and proximal segments of the screw 600 can be different (e.g. second thread form 625 more coarse than the first thread form 615) such that the screw can cause compression across the sacroiliac joint. The compacted cancellous bone as well as the insertion of at least a portion of the screw 600 into the burred defect in the anterior sacral cortex each aid in the optimization of the screw purchase.

Material can be advanced into at least a portion of the cannulated bore 640 of the screw 600. The material advanced into the cannulated bore 640 can vary including, but not limited to, bone graft or bone graft substitutes, cancellous bone or other osteo-proliferative, and/or osteo-inductive, and/or osteo-conductive material. The material can be in a dowel or cylindrical form. The material can be inserted from a proximal to distal direction (i.e. the direction of screw advancement). The material can be inserted before, during, and/or after installing the screw 600 into the previously-prepared channel. The bone material can be advanced under force or pressurization through the proximal cannulation 640 of the screw 600. A delivery element can be used to advance the material into the cannulated bore 640. The delivery element can be configured to couple with the screw 600 itself in order to maintain a net-zero displacement force on the screw 600 as the material is advanced into the cannulated bore 640. The coupling mechanism between the delivery element and the screw 600 can vary including, but not limited to thread forms, undercuts or other features configured to mate together. For example, the delivery element can be a threaded advancing screw configured to engage an internal feature 642 such as female threads within the cannulated bore 640 near the proximal segment 610 of the screw 600 (best shown in FIG. 6C). The internal feature 642 can allow for advancing material from a proximal end region toward a distal end region of the screw 600. Alternatively or additionally, a cannula can be linked to the proximal segment 610 of the screw 600 via an external feature 643 such as a groove, shelf or undercut that resists a proximal pulling load on the screw 600 (best shown in FIGS. 6B and 6C). A plunger can be linked to the cannula or a threaded advancing screw positioned within the cannula, and used to advance and pressurize the material within the screw 600. The net-zero effect can be achieved by linking the forward displacing force of the delivery element (i.e. a plunger or advancing screw) to an equal and opposite reactive force on the screw 600 for example via the coupling mechanism (i.e. corresponding threads, undercut or other coupling features). The material advanced into the cannulated bore 640 can be advanced along the longitudinal axis of the screw 600 and in close proximity to or extruded through at least one fenestration 635, 645 within the sacroiliac screw 100. The material can be placed in communication with or result in the extrusion of such material outside the internal boundaries of the screw 600.

The devices described herein can be constructed of one or more biocompatible material or materials. The dual pitch sacroiliac screws 100, 600 including the central segment 130, 630 can be formed of relatively radiolucent material (such as PEEK, Carbon Fiber Impregnated PEEK, or ceramics, such as alumina or zirconia) allowing for improved radiographic intraoperative and postoperative visualization of sacroiliac joint arthrodesis progress. In a one segmented or composite implementation, the threaded portions of the sacroiliac screw can be constructed from radio-dense material (e.g. titanium or cobalt-chrome alloys). The entire sacroiliac screw can be molded or machined from radiolucent or relatively radiolucent material(s) and can be in part or in whole, surface coated with material(s) to promote osteo-integration or bone ingrowth (when porous) and otherwise on-growth (e.g. titanium and titanium alloys and hydroxyapatite). Nearly monolithic and all titanium alloy. The conical graft material radial dispersion element can be radiolucent PEEK polymer.

While this specification contains many specifics, these should not be construed as limitations on the scope of what is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results. Only a few examples and implementations are disclosed. Variations, modifications and enhancements to the described examples and implementations and other implementations may be made based on what is disclosed.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

What is claimed is:

1. A method of sacroiliac joint arthrodesis comprising:
placing a first guide pin within a sacroiliac joint, wherein the first guide pin is blunt-tipped and flexible such that the first guide pin takes a non-linear course along at least a portion of the sacroiliac joint;
inserting a reamer over the first guide pin, the reamer having a flexible and cannulated shaft configured to follow the non-linear course taken by the first guide pin;
reaming articular soft tissue and periarticular bone from both the ilium and the sacrum using the reamer and collecting reamed material within the shaft;
removing the reamer and the reamed material resulting in a non-linear void coursing generally along the at least a portion of the sacroiliac joint; and
advancing material into the non-linear void.

2. The method of claim 1, wherein the first guide pin is inserted between subchondral or periarticular plates of the sacroiliac joint from a relatively posterior-medial to a relatively anterior-lateral direction.

3. The method of claim 2, wherein the first guide pin is deflected by higher density periarticular bone and remains generally within the sacroiliac joint composed of relatively lower density articular soft tissue.

4. The method of claim 3, wherein the first guide pin is inserted in a percutaneous manner using fluoroscopic visualization through a cannulated guide.

5. The method of claim 1, further comprising:
inserting an obturator having a shaft extending through a thin-walled cannula to at least a posterior extent of the non-linear void, wherein the shaft of the obturator and the thin-walled cannula are flexible and configured to follow the non-linear course taken by the first guide pin; and
removing the obturator leaving the thin-walled cannula in position,
wherein advancing material into the non-linear void comprises advancing the material through the thin-walled cannula.

6. The method of claim 5, wherein the material is advanced from within the thin-walled cannula while the thin-walled cannula is withdrawn from the non-linear void.

7. The method of claim 1, wherein the material advanced into the non-linear void is selected from the group consisting of bone graft, bone graft substitute, cancellous bone, osteo-proliferative material, osteo-inductive material, and osteo-conductive material.

8. The method of claim 1, further comprising fixing the sacroiliac joint with a fixation element with or without compression.

9. The method of claim 8, wherein fixing the sacroiliac joint comprises advancing a generally linear guide pin in a posterior-lateral to anterior-medial direction and placing the linear guide pin across at least a portion of the sacroiliac joint.

10. The method of claim 9, wherein the linear guide pin is advanced from an osseous entry point, just caudal and lateral to the posterior superior lateral iliac ala, through the ilium and the sacroiliac joint and into the sacral ala and body, cranial to the first sacral foramen and caudal to the lumbosacral disc space on the ipsilateral side of the sacrum.

11. The method of claim 9, further comprising exchanging the linear guide pin with a blunt guide pin toward, but not through the anterior sacral cortex along a vector that intersects the anterior sacral cortex between the mid-sagittal plane and a parasagittal plane generally defined by medial margins of the sacral foramina.

12. The method of claim 11, further comprising inserting a cannulated reamer over the blunt guide pin to create a generally linear tract through the ilium, sacroiliac joint and a portion of the sacrum.

13. The method of claim 12, further comprising:
advancing at least one cannulated dilator tool over the blunt guide pin into the body of the sacrum but not through the anterior wall of the sacral body; and
inserting the fixation screw for fixation.

14. The method of claim 13, further comprising advancing a cannulated burr through the anterior wall of the sacral body, forming an anterior sacral cortical defect lateral to the mid-sagittal plane and medial to the sacral foramina.

15. The method of claim 14, wherein the cannulated burr is relatively blunt in profile, has fine teeth or a diamond dust coated surface with or without a depth stop to minimize risk of injuring soft tissue structures anterior the sacrum.

16. The method of claim 14, wherein the fixation screw comprises:
a proximal segment having a first thread form having a first pitch;
a distal segment having a second thread form having a second pitch that is coarser than the first pitch;
an intervening, non-threaded central segment;
a cannulated bore extending through at least a portion of the distal segment of the fixation screw; and
a blunt, non-threaded post projecting from the distal segment, wherein the post is configured to insert through the anterior sacral cortical defect.

17. The method of claim 16, further comprising inserting the distal segment of the fixation screw just short of the anterior sacral cortex and leaving the proximal segment of the fixation screw immediately adjacent to the posterior/lateral iliac cortex, wherein the first thread form of the proximal segment engages the ilium and the second thread form of the distal segment engages the sacrum and wherein the intervening non-threaded central segment spans the articular margins.

18. The method of claim 16, further comprising advancing material into at least a portion of the cannulated bore, the material selected from the group consisting of cancellous bone, osteo-proliferative, osteo-inductive, and/or osteo-conductive material.

19. The method of claim 18, wherein advancing material comprises using a delivery element configured to couple with at least a portion of the proximal segment of the fixation screw to maintain net-zero displacement force on the fixation screw as the material is advanced into the cannulated bore.

20. The method of claim 13, wherein the fixation screw is radiolucent for improved radiographic fusion healing assessment.
